(19) United States
(12) Patent Application Publication
(10) Pub. No.: US 2006/0093668 A1
(43) Pub. Date: May 4, 2006

(54) Melt granulated composition and modified release dosage form prepared from said composition

(75) Inventors: Michiel Onne Elema, Kobnhavn (DK); Per Holm, Vanlose (DK)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 11/305,682

(22) Filed: Dec. 16, 2005

Related U.S. Application Data
(63) Continuation of application No. 10/706,886, filed on Nov. 6, 2003.
Application No. 10/706,886 is a continuation of application No. 10/106,805, filed on Mar. 25, 2002, now abandoned.
Application No. 10/106,805 is a continuation of application No. PCT/DK00/00533, filed on Sep. 28, 2000.

(30) Foreign Application Priority Data
Sep. 28, 1999 (DK) PA 1999 01376

(51) Int. Cl. A61K 9/20

(52) U.S. Cl. 424/464

(57) ABSTRACT

Solid modified release dosage forms, prepared from melt granulated compositions comprising (A) one or more hydrophilic cellulose ether polymers, (B) a hydrophilic melt binder, and (C) a therapeutically active ingredient.

The present invention relates to melt granulated compositions comprising hydrophilic cellulose ether polymers, such as for example, hydroxypropyl methylcellulose as a carrier base material, binder or matrix system, and a hydrophilic melt binder. The melt granulated compositions according to the invention are useful for the preparation of solid modified release dosage forms.

Modified release pharmaceutical preparations have many advantages from a medical viewpoint, i.e. reduction of administration times, better compliance, decrease of side effects and the retention of effective concentration of medical material in the blood.

Hydrophilic cellulose ether polymers such as hydroxypropyl methyl cellulose are particularly useful for the preparation of modified release dosage forms of the matrix type.

The hydrophilic cellulose ether polymer functions as a binder or matrix system to regulate release of components from the dosage form. When administered to a subject and exposed to water, the polymer partially hydrates to form a gelatinous layer, which retard outward diffusion of the therapeutically active ingredient. This original protective gel layer, once formed, permits additional fluid to penetrate into the interior of the dosage device. As the outer gel layer begins to fully hydrate and dissolve, a new layer, which optimally is sufficiently strong to continue to retard outward diffusion, replaces it and thus maintains the modified release features of the dosage form, for the desired length of time.

In order to prepare solid, shaped dosage forms from fine particles or powders, it is generally necessary to process the powders in a manner to improve their flowability thereby enabling the resulting material to be compressed into tablets, encapsulated or molded.

Various granulation processes for improving flowability of powders or other particulate materials, in which the primary particles are bound together with a binder material into larger free-flowing agglomerates or granules, are well known in the art.

One such method is the “wet” granulation process, which is characterised in that the powders are combined with a binder material and moistened with water or an organic solvent under conditions which result in the formation of a wet granulated mass from which the solvent must then be evaporated. The wet granulation process, while widely employed, has certain recognised limitations arising from the use when necessary of non-aqueous solvents which are environmentally deleterious, and furthermore may not be readily adaptable in connection with moisture sensitive medicaments. In particular, wet granulation would not be suitable for granulation of hydrophilic cellulose ether polymers.

An alternative to the “wet” granulation processes is the melt granulation process which comprises the use of room temperature solid or semi-solid materials having a relatively low softening or melting range to promote granulation of powdered or particulate materials, in the substantial absence of added water or other liquid solvents. The low melting solids, when heated to a temperature at or near or the melting range, liquify to act as a binder or granulating medium, which spreads itself over the surface of powdered or particulate materials with which it is associated, and on cooling, forms a solid granulated mass in which the powder or particulate starting materials are bound. An advantage of the melt granulation technique is that it is a “one-pot” granulating technique.

The melt granulated compositions according to the invention are characterised in that the melt binder used for granulation of the hydrophilic cellulose ether polymer is a hydrophilic melt binder, such as polyethylene glycol.

To our knowledge, the only prior art disclosing the use of PEG in the granulation medium for the preparation of modified release formulations is U.S. Pat. No. 5,403,593. The patent discloses modified release formulations based on a hydrophilic cellulose ether polymer matrix, which has undergone melt granulation, using a mixture of a lipid and PEG as a melt binder.

It has now been found that solid modified release dosage forms of the matrix type can be prepared from a melt granulated composition of a hydrophilic cellulose ether polymer where a hydrophilic melt binder, such as polyethylene glycol, is used without the addition of a lipid as a melt binder. Surprisingly, the use of a hydrophilic melt binder alone does not alter the release profile of the matrix tablets.

### SUMMARY

The present invention thus relates, alone or in combination, to:

A melt granulated, substantially homogeneous compositions comprising:
(A) one or more hydrophilic cellulose ether polymers;
(B) a hydrophilic melt binder, and
(C) a therapeutically active medicament.

Hydrophilic melt binders include, but are not limited to, polyether glycols such as polyethylene glycol and polypropylene glycol, and polyethylene glycol esters or acids, as well as polyoxypropylene and polyethylene oxide, copolymers thereof and mixtures of the foregoing.

In a particular embodiment of the invention, the hydrophilic melt binder is a polyethylene glycol.

Suitably, the polyethylene glycol used as granulation medium or melt binder has an average molecular weight of about 3,000 to about 9,000. In a particular embodiment, the polyethylene glycol used as a granulation medium is selected from the group consisting of PEG 3000, PEG 4000, PEG 6000, or PEG 8000.

The term hydrophilic cellulose ether polymer includes but is not limited to hydroxypropylmethylcellulose, methylcellulose, hydroxypropylcellulose, hydroxyethylcellulose, sodiumcarboxymethylcellulose, Carbomer, carboxymethylhydroxyethylcellulose, as well as mixtures thereof.

Suitably the hydrophilic cellulose ether polymer used is hydroxypropylmethylcellulose, methylcellulose, or mixtures thereof.

According to a particular embodiment of the invention, the hydrophilic cellulose ether polymer is hydroxypropylmethylcellulose.

The hydroxypropylmethylcellulose used according to the invention may have a methoxyl content of about 19 to 30 wt % and a hydroxypropoxyl content of about 4 to 12 wt. %.

The composition of the invention may also contain various conventional excipients such as binders, diluents, disintegrants, lubricants, etc.

Such diluent or binder materials may be selected from lactose, starches, sodium alginate, dicalcium phosphate (hydrate), sugars, acacia, agar, calcium carrageenan, alginic acid, algin, agarose powder, microcrystalline cellulose, collagen, colloidal magnesium silicate, colloidal silicon dioxide, pectin, gelatin, calcium sulfate, ethyl cellulose and polyacrylates

In one embodiment of the invention, the composition according to the invention comprises:
(A) 10 to 75 wt. % of a hydrophilic cellulose ether polymer or a mixture of hydrophilic cellulose ether polymers
(B) 10 to 40 wt. % of a hydrophilic melt binder,
(C) a therapeutically active medicament.

In a particular embodiment, the melt granulated composition according to the invention comprises
(A) 10 to 60 wt. % of hydrophilic cellulose ether polymer or a mixture of hydrophilic cellulose ether polymers
(B) 10 to 30 wt. % of a hydrophilic melt binder,
(C) a therapeutically active medicament.

In a particular embodiment, the melt granulated composition according to the invention comprises
(A) 20 to 60 wt. % of hydroxypropylmethylcellulose;
(B) 10 to 20 wt. % of a hydrophilic melt binder; and
(C) a therapeutically active medicament.

In a further embodiment, the present invention relates to a solid modified release dosage form prepared by compression of a composition as above, suitably to a tablet.

The composition according to the invention may also be filled in capsules.

The modified release dosage form according to the invention may be coated with a membrane, i.e enteric coatings, coatings for masking taste, coloured coatings etc., using conventional techniques and coating materials.

In a final embodiment, the present invention relates to a process for preparing a melt granulated composition as above, which comprises:

(I) applying heat to a composition comprising:

(A) one or more hydrophilic cellulose ether polymers;
(B) a granulating medium comprising polyethylene glycol; and
(C) a therapeutically active medicament,
   (II) mixing the mass to provide a substantially homogeneous composition; and
   (III) cooling the composition to room temperature.

The hydrophilic cellulose ether polymers are well-known and are commercially available under several trade names. The grades available under a given trade name represent variations in composition and molecular weight. For example, commercially available hydroxypropyl methylcellulose (HPMC) comprises a series of compounds (Methocel E, F and K, and Metolose SH of Shin-Etsu, Ltd) each having a different chemical composition, within a methoxyl content range of about 19 to 30 wt. % and a hydroxypropoxyl content range of about 4 to 12 wt. %, and each being available in various viscosity grades.

Particularly useful according to the present invention are the relatively high viscosity grades of HPMC, e.g., the 4,000 cps grades of Methocel E and Metolose 60 SH; the 4,000 cps grade of Methocel F and Metolose 65 SH; the 4,000, 15,000 and 100,000 cps viscosity grades of Methocel K; and the 4,000, 15,000, 39,000 and 100.000 grades of Metolose 90 SH.

Preferred are Metolose 90SH-15.000 and Metolose 90SH-100.000

Examples of methylcellulose polymers useful according to the invention comprise Methocel A of The Dow Chemical Co., and Metolose SM of Shin-Etsu, Ltd., having a methoxyl content of about 27.5-31.5 wt. %.

Particularly preferred are the higher viscosity grades including the 4,000 and 15,000 cps viscosity grades of Methocel A and Metolose SM and the 4,000 cps viscosity grade of Methocel A4M.

Specific examples of suitable methylcellulose polymers comprise Methocel A15LV, Methocel A4C, Methocel A15C and Methocel A4M of the Dow Chemical Co. (see, e.g., METHOCEL Cellulose Ethers: Technical Handbook, The Dow Chemical Co,. c. 1988).

The foregoing HPMC and methylcellulose polymers can be used individually or in combination in the compositions of the invention.

Other potentially suitable hydrophilic cellulose ether polymers comprise hydroxypropylcellulose, hydroxyethylcellulose, sodiumcarboxymethylcellulose, carboxypolymethylene, carboxymethylhydroxyethylcellulose, etc.

Finally, certain cellulose ether based formulations useful according to the invention for the preparation of modified release dosage forms are exemplified in U.S. Pat. Nos. 3,065,143, 3,870,790, 4,226,849, 4,357,469, 4,389,393, 4,540,566, 4,795,327 and 4,849,229.

Suitably, component (B) of the compositions of the invention is a hydrophilic melt binder composed of a hydrophilic melt binder having a melting range above 30° C.

Typically, the hydrophilic melt binder has a melting range above 40° C. The melt binder preferably has a melting range within the range of about 50° C. to 100° C., and more preferably within the range of about 60° C.-70° C.

Hydrophilic melt binders include, but are not limited to, polyether glycols such as polyethylene glycol and polypropylene glycol, and polyethylene glycol esters or acids, as well as polyoxypropylene and polyethylene oxide, copolymers thereof, and mixtures of the foregoing, xylitol, acrylate polymers (polymethacrylate).

Polyethylene glycol (PEG) has the generalised formula HO—(CH₂CH₂O)ₙ—H, wherein n represents the average number of oxyethylene groups. PEG, in its commercial forms, generally has a designation corresponding to the average molecular weight of the polymer.

PEG 3000 has a melting point around 48-54° C.; PEG 4000 has a melting point around 50-58° C., PEG 6000 has a melting point around 55-63° C. and PEG 8000, which is a waxy solid at room temperature, has a melting range around 60-63° C.

While any of the commercially available forms of PEG, which have a melting range above 30° C., are potentially suitable to prepare formulations of the invention, depending on the ultimate dosage form to be prepared, the preferred PEG polymers for use in the present invention comprise those having an average molecular weight of about 3,000 to about 9,000.

The compositions of the invention may also include various conventional excipients such as binders, diluents, disintegrants, lubricants, etc. well-known to the art.

Examples of conventional diluent or binder materials include lactose, starches, sodium alginate, dicalcium phosphate hydrate, sugars, acacia, agar, calcium carrageenan, alginic acid, algin, agarose powder, microcrystalline cellulose, collagen, colloidal magnesium silicate, colloidal silicon dioxide, pectin, gelatin, calcium sulfate, ethyl cellulose, polyacrylates, etc.

Examples of lubricants include talc, colloidal silicon dioxide, stearic acid, metallic stearates, hydrogenated vegetable oils, corn starch, polyethylene glycols, sodium benzoate and sodium acetate.

The granulating medium or melt binder can to an extent also acts as a lubricant in the composition. The amount of lubricant added to the total composition depend on the type of lubricant used. Typically, the lubricant is provided in an amount of about 1-2 wt. % (based on the total composition).

Other optional components of the composition according to the invention are colorants, sweeteners, etc.

Where appropriate, coatings may also be applied to the dosage forms of the invention, provided these are not incompatible with the desired modified release effect.

In order to prepare the melt granulated compositions of the invention, the components are heated to a temperature at which the granulation medium is at least partially in a molten state.

As the granulating medium is heated and becomes molten, it forms liquid bridges between the particles of the composition, which change to solid bonds upon cooling. A composition is thereby formed in which the granulating medium and the remaining components of the composition are closely bound together, forming agglomerates or granules of primary particles.

Preferably, the granulating medium is dry-blended with the hydrophilic cellulose ether component, the therapeutic agent and any other excipients (e.g., a solid diluent such as lactose) prior to heating. Alternatively, the granulating medium can be pre-heated to at least a softened state, and then combined with other components of the composition (HPMC and the therapeutic agent), and heat may be continued to be supplied to the resulting composition, for as long as necessary to carry out the melt granulation.

The compositions are maintained at an elevated temperature and blended for a time sufficient to form a substantially homogenous granulated product.

The resulting composition is then cooled, or allowed to cool, to room temperature. Suitably, the granulated product is gently blended while cooling.

In carrying out the process of the invention, heat may be applied by means of steam heat in a jacketed bowl equipped with blending means. For initial cooling, cold water may then be circulated through the jacketed bowl until the temperature drops below the melting range. The solidified material may then be recovered, optionally sized and allowed to cool to room temperature.

Alternatively, the granulate is passed through a sieve while still hot and thereafter allowed to cool to room temperature.

An alternative technique for preparing the melt granulated compositions of the invention, the molten granulating medium may be sprayed on a bed, which is preferably fluidized, comprising the remaining components of the compositions of the invention, e.g., the hydrophilic cellulose ether polymer, the therapeutic agent etc., under conditions suitable to form a melt granulated product. Alternatively, the solid granulation medium is blended with the remaining components of the compositions before loading into the fluid bed reactor. Granulation is thereafter carried out using hot air to form the fluidized bed.

The recovered melt granulated product can thereafter be screened, if necessary, and additional excipients, such as lubricants, may be added.

The resulting formulation is thereafter compressed, moulded, encapsulated or otherwise employed to result in formation of a solid modified release dosage form, using conventional techniques.

The composition according to the invention may be added or mixed with conventional excipients such as binders, diluents, disintegrants, lubricants, etc. as defined above, before compression, moulding or encapsulation to form a solid modified release dosage form.

The medicaments to be delivered using the modified release dosage for of the invention include inorganic and organic drugs.

In the examples below, compositions according to the invention has been prepared which comprise Citalopram, Escitalopram and Gaboxadole.

The examples are merely intended to illustrate the invention.

### EXAMPLE 1

Lactose monohydrate was combined with Metolose, Citalopram, HBr and Macrogol in a heat jacketed high shear mixer (Pellmix 1/8). The temperature regulator of the mixer was set to 80° C. and the combined ingredients were blended at 1200 rpm until the product temperature reached about 70° C. Granulation was continued for 1 to 2 minutes.

Immediately after granulation, the still hot granulate was passed through a 1 mm sieve. Sieving (1 mm sieve) was repeated after cooling. Magnesium stearate was added to the granulate in a Turbulate mixer and blended for 30 s.

The composition of the granulate after addition of magnesium stearate as a lubricant, is given in the table below. The batch size was 1 kg.

TABLE 1
Batch No. EX1131-20/EX1131-21

| Component | % w/w |
|---|---|
| Citalopram, HBr | 20 |
| PEG 6000 | 20 |
| Metolose 90SH-15.000 | 40 |
| Lactose 350 mesh | 19.5 |
| Magnesiumstearat (lubricant) | 0.5 |

The granulated product was loaded into a Korsh EK0 tabletting machine mounted with 7 mm convex punches and pressed into tablets.

The properties of the resulting tablets are given in the table below:

TABLE 2

| Property | Pressure (2 kN) | Pressure (21 kN) |
|---|---|---|
| Hardness, mean (N) | 33 N | 64 N |
| Mass, mean (mg) | 172.2 mg | 120.5 mg |
| Friability (Erweka) | 1.17% | 0.84% |

### EXAMPLE 2

Lactose monohydrate was combined with Metolose, Escitalopram, oxalate and Macrogol in a heat jacketed high shear mixer (Pellmix 1/8). The temperature regulator of the mixer was set to 80° C. and the combined ingredients were blended at 1200 rpm until the product temperature reached about 70° C. Granulation was continued for 1 to 2 minutes. Immediately after granulation, the still hot granulate was passed through a 1 mm sieve. Sieving (1 mm sieve) was repeated after cooling. Magnesium stearate was added to the granulate in a Turbulate mixer and blended for 30 s.

The composition of the granulate after addition of lubricant is given in the table below. The batch size was 1 kg.

TABLE 3
Batch No. EX1153-1/EX1153-2

| Component | % w/w |
|---|---|
| Escitalopram, oxalat | 10.2 |
| PEG 6000 | 20 |
| Metolose 90SH-15.000 | 40 |
| Lactose 350 mesh | 28.8 |
| Magnesuimstearat (lubricant) | 1.0 |

The granulated product was loaded into a Korsh EK0 tabletting machine mounted with 7 mm convex punches and pressed into tablets.

The properties of the resulting tablets are given in the table below:

TABLE 4

| Property | Pressure (2 kN) | Pressure (29 N) |
|---|---|---|
| Hardness, mean (N) | 38 N | 66 N |
| Mass, mean (mg) | 130.3 mg | 127.8 mg |
| Friability (Erweka) | 0.90% | 1.00% |

### EXAMPLE 3

Lactose monohydrate was combined with Metolose, Gaboxadol HCL and Macrogol in a heat jacketed high shear mixer (Pellmix 1/8). The temperature regulator of the mixer was set to 80° C. and the combined ingredients were blended at 1200 rpm until the product temperature reached about 70° C. Granulation was continued for 1 to 2 minutes.

Immediately after granulation, the still hot granulate was passed through a 1 mm sieve. Sieving (1 mm sieve) was repeated after cooling. Magnesium stearate was added to the granulate in a Turbulate mixer and blended for 30 s.

The composition of the granulate after addition of lubricant. The batch size was 1 kg.

TABLE 5
Batch No. EX1157

| Component | % w/w |
|---|---|
| Gaboxadol, HCl | 20 |
| PEG 6000 | 20 |
| Metolose 90SH-15.000 | 40 |
| Lactose 350 mesh | 19.5 |
| Magnesiumstearat (lubricant) | 0.5 |

The granulated product was loaded into a Korch EK0 tabletting machine mounted with 7 mm convex punches and pressed into tablets.

The properties of the resulting tablets are given in the table below:

TABLE 6

| Property | Pressure (32 kN) |
|---|---|
| Hardness, mean (N) | 35 |
| Mass, mean (mg) | 127.0 mg |
| Friability (Erweka) | 1.20% |

### EXAMPLE 4

The release properties of the tablets prepared according to example 1 and 2 were measured using the US paddle method with the modification that a peak bowl was used in order to restrain the tablet from settling in the centre of the bowl. The release profile of the tablets prepared according to example 3 was measured using the standard US paddle method.

The test conditions were as follows:

| | |
|---|---|
| Medium: | Phosphate buffer pH = 6.0 |
| Volume: | 900 ml |
| Revolutions: | 50 rpm |
| Bowl: | Peak type |

Accompanying FIGS. 1, 2 and 3 show the release from the tablets prepared according to example 1, 2 and 3.

It will be seen that the tablets of the invention provide a modified release of the medicament over an extended period of time.

1. A melt granulated substantially homogeneous composition comprising:
(A) one or more hydrophilic cellulose ether polymers;
(B) a hydrophilic melt binder; and
(C) gaboxadol.

2. A composition according to claim 1 wherein the hydrophilic melt binder is a polyethylene glycol.

3. A composition according to claim 2 wherein the polyethylene glycol has an average molecular weight of about 3,000 to about 9,000.

4. A composition according to claim 1 wherein the hydrophilic cellulose ether polymer is selected from the group consisting of hydroxypropylmethylcellulose, methylcellulose, hydroxypropylcellulose, hydroxyethylcellulose, sodiumcarboxymethylcellulose, Carbomer, carboxymethylhydroxyethylcellulose and mixtures thereof.

5. A composition according to claim 4 wherein the hydrophilic cellulose ether polymer is hydroxypropylmethylcellulose, methylcellulose, or mixtures thereof.

6. A composition according to claim 5 wherein the hydrophilic cellulose ether polymer is hydroxypropylmethylcellulose.

7. A composition according to claim 1 further comprising an excipient.

8. A composition of claim 7 wherein said excipient is selected from the group consisting of a binder, diluent, disintegrant or lubricant.

9. A composition according to claim 7 comprising a diluent or binder, wherein the diluent or binder is selected from the group consisting of lactose, starches, sodium alginate, dicalcium phosphate hydrate, sugars, acacia, agar, calcium carrageenan, alginic acid, algin, agarose powder, microcrystalline cellulose, collagen, colloidal magnesium silicate, colloidal silicon dioxide, pectin, gelatin, calcium sulfate, ethyl cellulose and polyacrylates.

10. A composition according to claim 1 comprising:
(A) 10 to 75 wt % of a hydrophilic cellulose ether polymer or a mixture of hydrophilic cellulose ether polymers;
(B) 10 to 40 wt % of a hydrophilic melt binder; and
(C) gaboxadol.

11. A composition according to claim 10 comprising:
(A) 10 to 60 wt % of a hydrophilic cellulose ether polymer or a mixture of hydrophilic cellulose ether polymers;
(B) 10 to 30 wt % of a hydrophilic melt binder; and
(C) gaboxadol.

12. A composition according to claim 11 comprising:
(A) 20 to 60 wt % of a hydroxypropylmethylcellulose polymer;
(B) 10 to 20 wt % of a hydrophilic melt binder; and
(C) gaboxadol.

13. A composition according to claim 10 wherein the hydrophilic melt binder is a polyethylene glycol (PEG) with an average molecular weight of about 3,000 to 9,000.

14-16. (canceled)

17. A solid modified release dosage form prepared by compression of a composition according to claim 1.

18. A modified release dosage form according to claim 17, further comprising an excipient.

19. A modified release dosage form according to claim 17, which is in the form of a tablet.

20. A process for preparing a therapeutically active composition according to claim 1 comprising:
(1) applying heat to a composition comprising:
(A) one or more hydrophilic cellulose ether polymers;
(B) a hydrophilic melt binder; and
(C) gaboxadol;
(2) mixing the mass to provide a substantially homogeneous composition; and
(3) cooling the composition to room temperature.
